(19) United States
(12) Patent Application Publication
(10) Pub. No.: US 2021/0287572 A1
(43) Pub. Date: Sep. 16, 2021

(54) Hybrid Medical Bleed Control Trauma Care Learning Device

(71) Applicants: Alfonse Dimino, Freehold, NJ (US); Michael J Dimino, Freehold, NJ (US); Michael C Dimino, Freehold, NJ (US)

(72) Inventors: Alfonse Dimino, Freehold, NJ (US); Michael J Dimino, Freehold, NJ (US); Michael C Dimino, Freehold, NJ (US)

(21) Appl. No.: 17/060,629

(22) Filed: Oct. 1, 2020

(51) Int. Cl. G09B 23/30

(52) U.S. Cl. CPC G09B 23/303; A61B 17/1322

(57) ABSTRACT

A learning device to teach trauma care consisting of a simulated limb to practice blood occlusion using a tourniquet in addition to wound packing and other medical techniques.

### BACKGROUND OF THE INVENTION

The present invention relates to teaching care of trauma wounds by means of but not limited to packing wounds with gauze, suturing, occluding blood flow with tourniquet and chest seals.

### SUMMARY OF INVENTION

The objective of the present invention is to provide learning device to teach wound packing, trauma assesment, and how to apply tourniquet and occlude blood flow; how to suture a laceration and the feel of an artery inside of a wound provides a realistic real-life scenario.

The first objective of the present invention is to simulate a real-life hemorrhage of an artery by applying a tourniquet properly on the side of the cylinder will close the artery inside of the wound hole and occlude the blood flow.

The second objective is to simulate the flow of blood through the artery(Hose) by use of the syringe forcing the blood flow through the cylinder and allowing it to leak out of the artery in the wound hole.

The third objective into teach how to pack a wound and stop the hemorrhage of the artery inside the wound hole by packing the wound with enough gauze that will stop the bleed artery.

The fourth objective is to teach suturing on lacerations on the base of the device.

The fifth objective to simulate the flow of blood. The simulated body part (Device) by use of syringe filled with blood and manual pushing the plunger down in one single push or pulsing the plunger with on and off motions, this simulated the pulsing of the flow if blood through the heart.

The sixth objective to simulate the flow of blood through the artery is to use a pump that is place inside a reservoir of simulated blood and setting a pulse action through a micro controller to turn the pump on/off—on/off. To simulate a heart pumping blood.

The seventh objective is to teach how to apply a chest seal. The needle acts as the lungs by pumping air through the tube, a chest seal is applied to the top of the wound hole by using the needle plunger pushing it into the needle back in an out again; simulates the movement of the lungs, thus, sucking the chest eal around the wound hole. The needle can be fitted with blood and allow to fill up wound hole and the blood will pass out the chest seal vent hole, allowing both air and blood to escape through vents.

### DETAIL DESCRIPTION OF DRAWINGS

Referring to the drawings FIG. 1 illustrates a learning device, when needle 6 is filled with a liquid. The liquid is injected into tube 5A (Simulated artery) which fill #3 simulated wound hole with liquid, when gauze is properly packed into #3 the bleeding will stop. This illustrates how a person can stop hemorrhaging wound, and how much gauze is needed to do so. #4 are simulated lacerations. The user can practice suturing the lacerations closed.

FIG. 2

#7 is a resource filled with liquid #8 is a pump that is submerged in the liquid. The liquid is pumped through the simulated artery hose into wound hole #3 which fills up with liquid until gauze packed into wound hole #3. The liquid continuously flows back into reservoir by return hose #12.

FIG. 3

Shows the learning device with chest seal #10 placed over wound hole #3. The purpose of the chest seal is to allow blood and air to escape through the vent holes 11 and 14, 13 shows the direction of air escaping. The chest seal prevents pneumothorax (the collapse of the lungs). When 5A is interfaced with a empty syringe, air can be injected into hose 5A which will cause chest seal to raise and air will escape through vent chambers 11&14 by pulling syringe plunger in reverse causing the effect of the lung taking in air.

### Description of Drawing

FIG. 1 shows the learning device.

#1 shows the side of the cylinder device.

#2 shows the top of the cylinder.

#3 shows the hole in the cylinder where wound packing is done.

#4 shows the base of the device where the user can practice suturing.

#5a is the simulated artery that protrudes out the base of the device.

#5b shows the artery on the inside of the wound the length of the device and crossing over wound packing hole.

#6 shows the syringe that pumps simulated flow of blood or fluid through the artery.

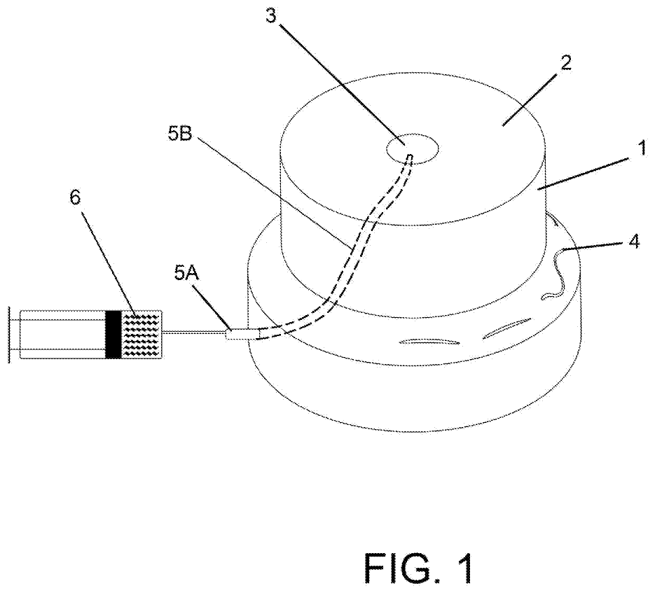

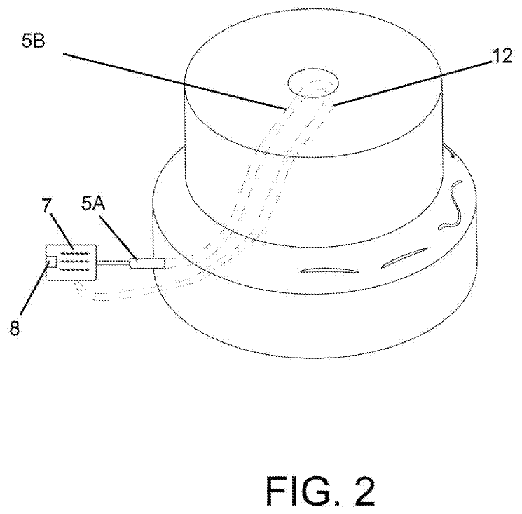

FIG. 2 #5A shows the simulated artery hose. #7 shows reservoir of water or simulated blood. #8 shows pulse motor that seats in water reservoir. #12 shows return host.

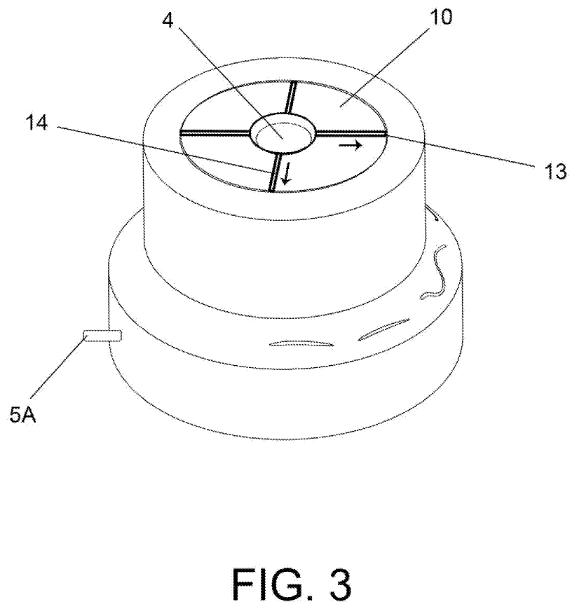

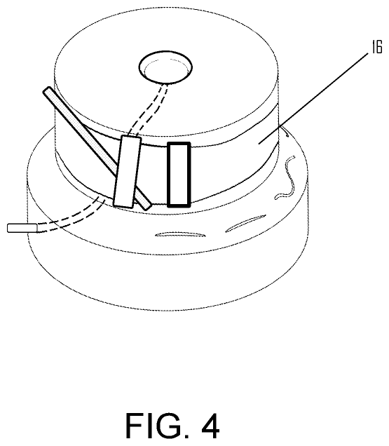

FIG. 3 #10 is the chest seal covering the wound hole #3. #11 is the vent hole of the chest seal. #13 is the blood and air escaping through the chest seal vent. #14 is the chest seal. #15 arrow indicates direction of the blood and air.

FIG. 4.

#16 shows Tourniquet or limb occlusion devices wrapping the Hybrid Medical Bleed Control Trauma Care Learning Device

What is claimed:

1. A portable learning device consisting of a simulated limb with multiple layers of the skin, including skin, muscle and fat; using material that looks and feels like skin.

2. According to claim 1, the device is shaped in the form of a cylinder mounted on a platform.

3. According to claim 2 the platform has laceration in order to demonstrate suturing the gap opening of the laceration; it can be seen and felt through different layers of the skin which also allows cosmetic suturing of different layers of the skin.

4. According to claim 2 the top of cylinder has a hole to simulate a gaping wound hole similar to a bullet hole or knife wound. This hole allows for training how to pack a wound with gauze.

5. According to claim 4, inside the hole can be seen and felt a simulated artery made tubing with a slight puncture mark through the artery to allow the flow of blood to escape the artery.

6. According to claim 5, the artery in the hole of the top cylinder is a tube that functions as an artery. The tube protrudes at the base of the device where a syringe can be attached to simulate blood flow traveling up the tube to the hole in the cylinder. This is where packing the wound is accomplished. If packing the wound is done properly with gauze the bleeding will stop.

7. According to claim 6, in addition to a syringe to pump blood, an electric pump can be used to pulse simulated blood or water or any fluid through tube.

8. According to claim 7, the pump can be controlled by an electric current that automatically pulses the pump on and off at a predetermined rate of speed/cycles.

9. According to claim 1 the learning device allows for a tourniquet to be placed on the top part of cylinder to teach how a tourniquet is used.

10. According to claim 9, the top part of the cylinder can be used to teach how much pressure is needed to occlude blood flow using a tourniquet. The tourniquet will pinch the artery and occlude blood flow in the artery(tube). If the correct amount of pressure is used the artery inside of the hole will stop bleeding thus simulating a real-life scenario of a hemorrhage.

11. According to claim 7 an electric pump sits inside a reservoir of simulated blood which pumps the blood through the artery. The pump is controlled by an electric circuitry.

12. According to claim 11, the electric pump is positioned inside a reservoir of simulated blood where the tube connects to tube on one end and the other goes through the learning device acting as a closed system of continuing the flow of blood.

13. According to claim 4, the gaping hole on top of cylinder can also be used to practice chest sealing to top of cylinder which will allow the blood flow to reach the top of cylinder hole, depending on type of chest seal used the blood in cylinder bill begin to escape slowly through the chest seal; thus demonstrating simulator situation of teaching control of pressure in chest when using a chest seal. The purpose of the chest seal is to allow blood to escape wound without letting air in that would cause lungs to collapse.
